(19) United States
(12) Patent Application Publication
(10) Pub. No.: US 2023/0364078 A1
(43) Pub. Date: Nov. 16, 2023

(54) METHOD OF PREPARING NANOPARTICLES COMPRISING POORLY WATER SOLUBLE DRUGS FOR TREATING HEPATITIS

(71) Applicant: EVEX Technology, Inc., Milpitas, CA (US)

(72) Inventors: Hyun Chul KIM, Gangdong-gu (KR); Byoung Yong MOON, Cupertino, CA (US)

(21) Appl. No.: 17/743,388

(22) Filed: May 12, 2022

(51) Int. Cl. A61K 31/4985; A61K 9/16; A61K 31/4178; A61K 31/4725; A61P 1/16

(52) U.S. Cl. CPC A61K 31/4985; A61K 9/1617; A61K 31/4178; A61K 31/4725; A61P 1/16

(57) ABSTRACT

The present invention provides nanoparticles which contain a poorly water-soluble drug for treating hepatitis, and a method of producing the nanoparticles. More particularly, the present invention provides a method of producing nanoparticles for treating hepatitis, which includes: a first step of preparing a precursor mixture solution for synthesis of nanoparticles which include a drug for treating hepatitis; a second step of extracting the compound under supercritical or subcritical conditions; and a third step of dispersing the same, as well as the nanoparticles including a poorly water-soluble drug for treating hepatitis with improved bioavailability and without organic solvent residue.

### BACKGROUND OF THE INVENTION

### 1. Field of the Invention

The present invention relates to a method of producing nanoparticles which contain a poorly water-soluble drug for treating hepatitis, and more particularly, to a method of producing nanoparticles which contain a poorly water-soluble drug with excellent particle homogeneity.

### 2. Description of the Related Art

The biopharmaceutical classification system (BCS) is a system that classifies drugs based on water solubility and intestinal permeability, and is widely used in academic, industrial and regulatory fields. BCS is divided into four stages: Class 1 has high solubility and permeability; Class 2 has low solubility but good permeability; Class 3 has high solubility but low permeability; and Class 4 has low solubility and permeability.

Quite a number of preparations contain poorly water-soluble or water-insoluble organic compounds as medicinal properties. Indeed, with regard to problems for development of new medicine in pharmaceutical industry, one of significant difficulties is poorly water-soluble property of candidate substances and many active pharmaceutical ingredients on the market are known to be poorly water-soluble. Such poor water-solubility limits absorption of active ingredients in a preparation into a living body, hence causing reduction in bioavailability and deterioration in medical efficacy.

Improving the bioavailability of a preparation to decrease a dose thereof and consequently reduce adverse effects on the living body is one of very important tasks in medical and pharmaceutical fields. In general, the bioavailability of a preparation is determined by physicochemical properties of the drug, the formulation and the route of administration. For example, oral formulations have advantages of being simple and less painful compared to injectable formulations (parenteral formulations), while involving a disadvantage of low bioavailability.

Oral preparations enter the intestine via the stomach and duodenum, and are mainly absorbed into the blood from the intestinal tract followed by going to the liver through the portal vein. While the oral preparation passes through these long pathways, some may be degraded under the action of gastric acid, etc., or may be metabolized in the liver to be changed into completely different substances. Low bioavailability of the oral preparation may be resulted from various factors such as: low dissolution rate; poor solubility; primary passing metabolism by the digestive tract and the liver; chemical instability in the stomach and the intestine; outflow transportation; and poor permeability through intestinal mucosa, which act alone or in combination with thereof. Further, a problem due to poorly water-soluble properties of active ingredients may also act in combination with the above factors.

Preparations including poorly water-soluble or water-insoluble organic compounds as active ingredients are typically administered to a living body by means of: a method of preparing the organic compound by dissolving the same in an organic solvent; a method of micronizing the organic compound into particles in micron unit and mixing the same with water or the like. However, the organic solvent for dissolving the organic compound may cause an undesired problem in a medical aspect, thus tending to turn away from using it.

Micronization of the organic compound is used as a means to improve solubility. When preparing microfine particles, high physical stability and homogeneity of the particles should be considered most importantly. This is because a uniform size and shape of the particles are directly associated with effects of a drug, and the microfine particles involve high possibility of easy coagulation. Currently, microfine particles prepared through milling, high pressure homogenization, precipitation, etc., which are widely used for micronization, have irregular sizes and shapes and deterioration in stability, and entail a problem of remaining the organic solvent used in the process.

Meanwhile, although poor bioavailability of a preparation is considered due to a dissolution problem, it could not be obviously determined whether the above problem is caused by low dissolution rate or low solubility level of a drug. Therefore, there is a limitation in expectation as a whole in that the bioavailability would be improved even when increasing the dissolution rate through micronization.

Hepatitis C Virus (hereinafter, “HCV”) is a virus as major causes for non-A or non-B hepatitis. HCV is known as a pathogenic factor of acute or chronic hepatitis, hepatocirrhosis and hepatic cancer. Treatment of HCV has greatly been developed along with development of several antiviral drugs since 2011.

Many of drugs used as therapeutics for chronic type C hepatitis show no or very little water-solubility. Daclatasvir is used as an antiviral drug, which is applicable to a combination therapy for treating chronic type C hepatitis as one of infectious liver diseases due to HCV infection. According to the biopharmaceutical classification system (BCS), the above preparation is a drug belonging to class 2, which is insoluble in water whereas being dissolved is an organic solvent DMSO. Alternatively, among other antiviral drugs for treatment of chronic type C hepatitis, Grazoprevir and Asunaprevir also belong to class 2, and similar to the above preparation, are very little soluble in water. Further, Velpatasvir used in combination with Sofosbuvir corresponds to a poorly water-soluble drug belonging to class 4.

### Prior Art Disclosure

Patent Document: KR 1020160117033 A

### SUMMARY OF THE INVENTION

An object of the present invention is to provide a pharmaceutical composition for treating hepatitis, with enhanced bioavailability by improving solubility and permeability of poorly water-soluble hepatitis therapeutics.

In addition, another object of the present invention is to provide a method of producing nanoparticles of poorly water-soluble hepatitis therapeutics.

Further, another object of the present invention is to provide a method of producing nanoparticles, which includes obtaining nanocrystals of poorly water-soluble hepatitis therapeutics with excellent stability and homogeneity using supercritical carbon dioxide or subcritical water under controlled temperature and pressure in the presence of vents and nozzles.

According to an aspect of the present invention, there is provided a method of producing nanoparticles for treating hepatitis, which includes: a first step of preparing a precursor mixture solution for synthesis of nanoparticles which include any one of compounds represented by Formulae 1 to 4 below; a second step of extracting the compound under supercritical or subcritical conditions; and a third step of dispersing the same.

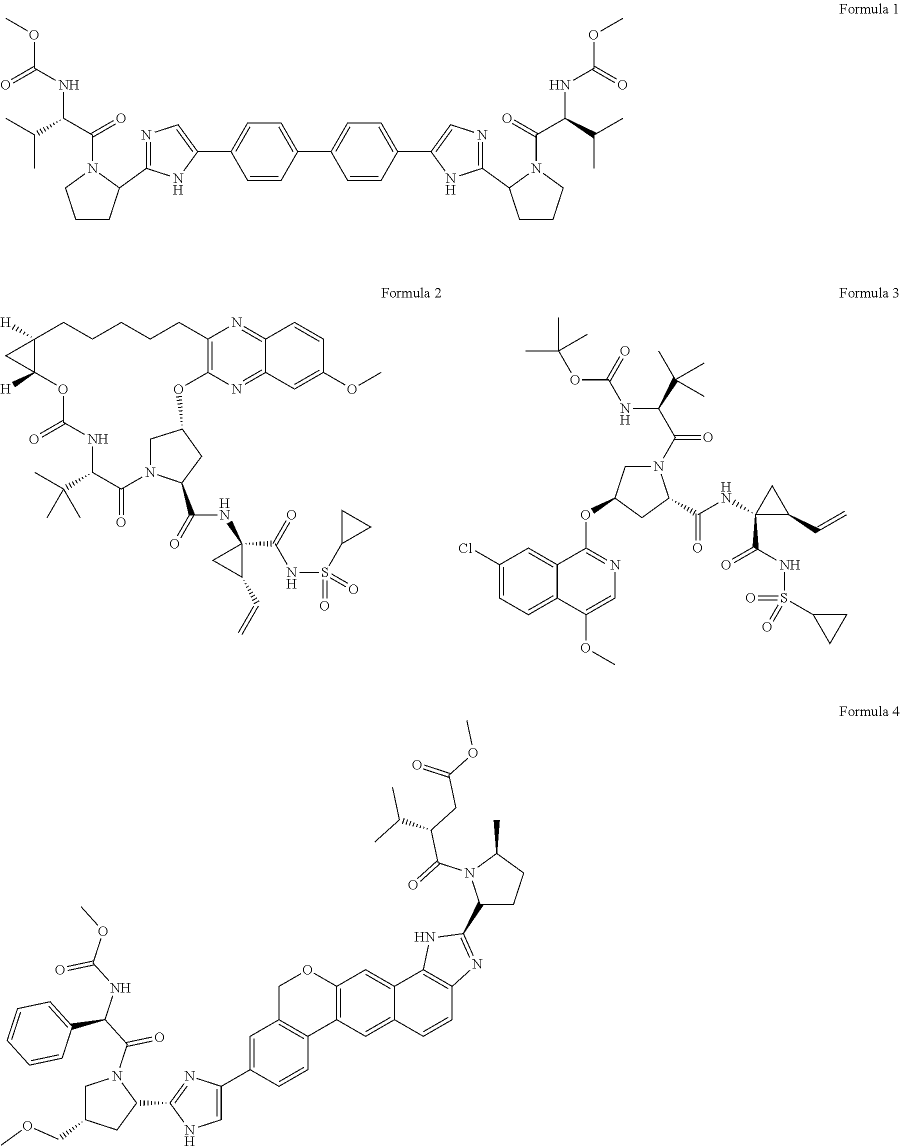

Further, the above precursor mixture solution may be a nanoemulsion or microemulsion including any one of the compounds represented by Formulae 1 to 4.

Further, the nanoemulsion or microemulsion may be prepared by mixing any one of the compounds represented by Formulae 1 to 4, oil, a surfactant and alcohol, wherein the oil, the surfactant and the alcohol may be included in a weight ratio value of 1 to 10 according to Equation 1 below, respectively:

(Weight ratio)=(A)/[(B)+(C)] Equation 1

(in Equation 1 above, A represents a weight of the oil, B represents a weight of the surfactant, and C represents a weight of the alcohol).

Further, the precursor mixture solution may be prepared by mixing any one of the compounds represented by Formulae 1 to 4, a surface modifier and a stabilizer in a solvent.

Further, the precursor mixture solution may be sequentially heated and cooled to produce a solid lipid matrix.

Further, the second step may be performed by adding the product prepared in the first step and an additive to a supercritical carbon dioxide fluid at 30 to 32° C., dissolving the same in the supercritical carbon dioxide fluid, and then extracting the same.

Furthermore, the second step may be performed by adding the product prepared in the first step and an additive to a subcritical water fluid at 110 to 140° C., dissolving the same in the subcritical water, and then extracting the same.

According to another aspect of the present invention, there are provide nanoparticles for treating hepatitis, which are prepared by the above production method.

According to an aspect of the present invention, it may be provided a method of producing nanoparticles, which can micronize a drug with excellent homogeneity and stability using solid lipid as a matrix. Through the above method, it is possible to produce a preparation with excellent bioavailability while improving the solubility of a poorly water-soluble preparation.

Further, since no transporter is required and an amount of excipient to be used is reduced, nanoparticles with enhanced loading capacity may be produced.

Further, it is possible to produce nanoparticles, which are surface-modified thus to have excellent stability, and enable active targeting similarly to a drug delivery system.

Further, denaturizing of medicines due to heat and pressure by a mechanical force, which possibly occurs in a conventional top-down way, is not caused, thereby solving a problem of organic solvent residues.

Further, it may be provided a method of producing nanoparticles, which enables eco-friendly production of a drug without organic solvent residues.

Furthermore, it may be provided a method of producing nanoparticles, which is not only implemented in a lap level but also may be easily scaled-up to a commercial scale.

### BRIEF DESCRIPTION OF THE DRAWINGS

The above and other objects, features and other advantages of the present invention will be more clearly understood from the following detailed description taken in conjunction with the accompanying drawing, in which:

FIG. 1 illustrates ethanol/oil emulsion state prepared according to an aspect of the present invention.

### DETAILED DESCRIPTION OF THE INVENTION

The present invention provides nanoparticles containing a poorly water-soluble drug for treating hepatitis, and a production method thereof.

In description of the present invention, the nanoparticles include nanocrystals other than nanosized microfine particles. The nanocrystals mean a stabilized shape of nanosized drug and has an advantage of improving drug dissolution rate and loading rate.

Hereinafter, the present invention will be described in more detail. In description of the present invention, publicly known techniques related to the present invention, which are judged to be able to make the purport of the present invention unnecessarily obscure, will not be described in detail.

According to an aspect of the present invention, there is provided a method of producing nanoparticles which include a poorly water-soluble drug for treating hepatitis after nanorization of the same.

With regard to the production method, firstly, a step of preparing a precursor mixture solution for synthesis of nanoparticles, wherein the precursor mixture solution contains a poorly water-soluble drug for treating hepatitis, may be performed (S1).

The precursor mixture solution may be prepared by mixing a drug for treating hepatitis, a surface modifier and a stabilizer in a solvent. The drug for treating hepatitis, the surface modifier and the stabilizer may be introduced sequentially or simultaneously, wherein the order of introduction can be adjusted on the basis of common knowledge in the art, but it is not limited thereto.

The drug for treating hepatitis may include a compound belonging to class 2 or 4 according to the classification of the biopharmaceutical classification system (BCS). According to a preferred embodiment of the present invention, the drug for treating hepatitis may include at least one compound selected from the group consisting of compounds represented by Formulae 1 to 4 below. The drug for treating hepatitis may include any one of Daclatasvir, Grazoprevir, Asunaprevir and Velpatasvir, but it is not limited thereto.

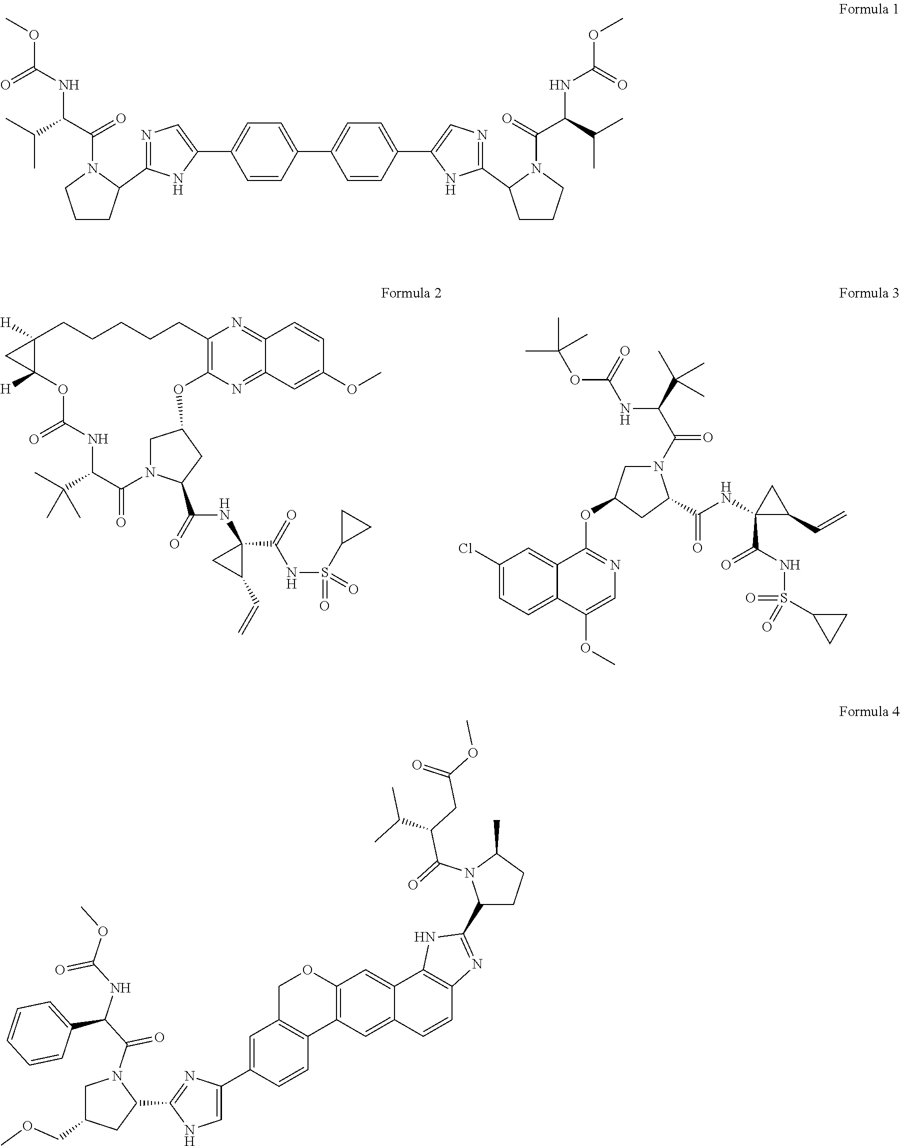

The surface modifier may contain lipid of fatty acid, and preferably, lipid. The lipid may include at least one selected from the group consisting of 1-hexadecanol, 1-pentadecanol and 1-tetradecanol, but it is not limited thereto. The surface modifier may be adsorbed to the drug for treating hepatitis in a content capable of maintaining an average particle size of the produced nanoparticles in a range of 800 to 5400 nm. When the particle diameter of the produced nanoparticles is less than 800 nm, handling of post-process is difficult due to coagulation. If the particle diameter exceeds 5400 nm, it may cause a problem in that desired effects are hardly achieved. Each surface modifier molecule adsorbed to the drug for treating hepatitis may be adjusted to have an intermolecular cross-linking rate at a very low level of less than 10%. For example, the cross-linking rate may be adjusted to 1 to 8%, but it is not limited thereto. When the cross-linking rate exceeds the above range, side reactions are increased in the process to be described below and features imparted by the corresponding process are inhibited and may cause a problem in that the desired effects are hardly achieved.

The stabilizer may be added to increase stability, and may include at least one selected from the group consisting of polyvinyl pyrrolidone and poloxamer 188, but it is not limited thereto.

The solvent may be selected from those capable of partially dissolving the drug for treating hepatitis. For example, at least one selected from the group consisting of alcohol, hexane, acetone, acetonitrile, cyclohexane, ethyl acetate, tetrahydrofuran (THF), formamide, dimethylformamide (DMF), dimethyl sulfoxide (DMSO) and cyclohexanone may be used, but it is not limited thereto. Preferably, the solvent is selected from water or alcohol. The alcohol used herein may include, for example, methanol, ethanol, propanol, isopropyl alcohol and liquid fatty alcohol, but it is not limited thereto. Preferably, the alcohol used herein is alcohol having 2 to 20 carbon atoms, and more preferably, ethanol is selected.

The drug for treating hepatitis, the surface modifier and the stabilizer included in the precursor mixture solution may be controlled to have a content in specific ratio by weight (“weight ratio”), respectively. The content of the drug for treating hepatitis may be adjusted in consideration of solubility to a solvent. For example, when the compound represented by Formula 1 is selected as a drug ingredient and ethanol is used as the solvent, a solubility of the compound represented by Formula 1 is 148 mg/ml. Therefore, the content of the drug may be controlled so that a mixture solution is prepared in a state of not excessively increasing a viscosity of the solution while dissolving the compound as much as possible in the solvent to be used. As another example, if the compound represented by Formula 2 is selected as a drug ingredient and ethanol is used as the solvent, a solubility of the compound represented by Formula 2 to ethanol is 15 mg/ml. Therefore, in consideration of this solubility, the content of the drug may be adjusted so as to prepare a mixture solution as described above. In addition, as another example, if the compound represented by Formula 3 is selected as a drug ingredient and ethanol is used as the solvent, a solubility of the compound represented by Formula 3 to ethanol is 100 mg/ml. Therefore, in consideration of this solubility, the content of the drug may be adjusted so as to prepare a mixture solution as described above. Further, as another example, if the compound represented by Formula 4 is selected as the drug ingredient and ethanol is used as the solvent, a solubility of the compound represented by Formula 4 to ethanol is 30 mg/ml. Therefore, in consideration of the solubility, the content of the drug may be of course adjusted so as to prepare a mixture solution as described above. A viscosity of the solution may be adjusted on the basis of technical common knowledge in the art, for example, in a range of 1 to 7 Pas, preferably, 1 to 5 Pas, and more preferably, 4 Pas or less.

The surface modifier may be mixed to be included in a content of 1 to 10% by weight (“wt. %”) based on a total 100 wt. % of the precursor mixture solution. When the content of the surface modifier is less than 1 wt. %, a precipitation or coagulation phenomenon may occur. If the content exceeds 10 wt. %, side reactions such as self-emulsification or a difficulty in purification after reaction may be caused. Preferably, the surface modifier may be included in an amount to be 1 to 5 wt. %, and more preferably, 2 to 3 wt. % based on the total 100 wt. % of the precursor mixture solution.

The stabilizer may be mixed so as to be included in a content of 0.1 to 0.5 wt. % based on the total 100 wt. % of the precursor mixture solution. When the content of the stabilizer is less than 0.1 wt. %, it is difficult to exhibit effects obtained by addition of the stabilizer. If the content of the stabilizer exceeds 0.5 wt. %, it may cause a difficulty in adjusting a particle size due to prevention of adsorption of the surface modifier.

If necessary, the precursor mixture solution may further include oil. The oil may be included so as to be a content of 60 to 95 wt. % based on the total 100 wt. % of the precursor mixture solution. When the oil content is less than 60 wt. %, the solution viscosity may be increased thus to cause problems of coagulation and precipitation. If the oil content exceeds 95 wt. %, it may cause a problem of decreasing economical advantage. Preferably, the oil content ranges from 90 to 95 wt. %.

If necessary, the precursor mixture solution may further include an additive. The additive may be selected, for example, from oxidants or reductants in order to improve purity of nanoparticles, but it is not limited thereto.

If necessary, the prepared precursor mixture solution may be subjected to heating and cooling. Solidification may be performed through heating and cooling, thus to form a solid lipid matrix.

The heating may be performed by raising a temperature until individual substances introduced to the precursor mixture solution are completely dissolved. At this time, agitation is preferably conducted.

The cooling may be sequentially performed after the heating, and may be conducted at room temperature. In this regard, the room temperature means a temperature in a range of 15 to 35° C. The cooling may be performed for 20 to 30 hours, and preferably, for 24 hours.

The precursor mixture solution prepared in the step S1 may be prepared in a state in which the drug is introduced in a solvent such as ethanol as described above, or otherwise, may be prepared in a state in which the drug is emulsified. During emulsification, the solution may be divided into a microemulsion or nanoemulsion state depending on an average size of the emulsified particles, and the microemulsion may allow the particles to have an average size of 50 to 200 nm, while the nanoemulsion may allow the particles to have an average size of 100 to 1000 nm.

A mixture for emulsification of the drug may include at least one selected from the group of the compounds represented by Formulae 1 to 4, oil, a surfactant, and alcohol having 2 to 20 carbon atoms. At this time, the oil (A), the surfactant (B) and the alcohol (C) having 2 to 20 carbon atoms may be included in the mixture so that they have a weight ratio value of 1 to 10 calculated by Equation 1 below:

(Weight ratio)=(A)/[(B)+(C)]. Equation 1

In Equation 1 above, A represents a weight of the oil, B represents a weight of the surfactant, and C represents a weight of the alcohol. When the weight ratio value in Equation 1 is included in the above range, an emulsion with excellent stability over time, wherein an average size of the emulsified particles ranges from 100 to 1000 nm, may be prepared.

If necessary, in order to improve stability of the emulsion, an emulsifier may be added in the mixture. The emulsifier is preferably selected from those with a low HLB value. For example, the emulsifier may be at least one selected from the group consisting of glyceryl esters, sugar esters, glucose esters and PEG esters, but it is not limited thereto.

The surfactant is preferably selected from those having HLB 3.5 to 6. For example, the surfactant may be at least one selected from the group consisting of polyglyceryl-4-diisostearate, glyceryl isostearate, sorbitan isostearate, methyl glucose dioleate and PEG-30-dipolyhydroxy stearate, but it is not limited thereto.

The emulsion may be prepared by heating an oil phase (O) and a water phase (W) to completely dissolve the same, slowly introducing W phase based on O phase to perform pre-emulsification, followed by secondary emulsification and cooling.

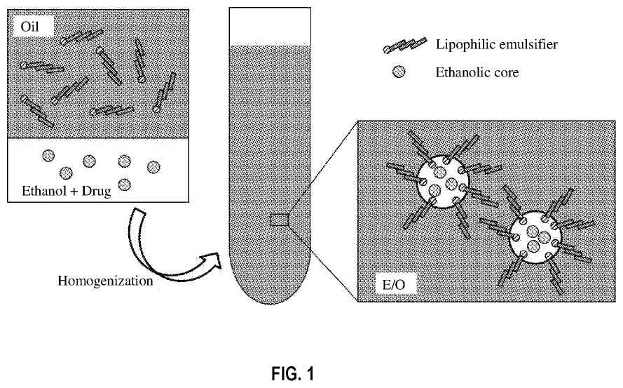

One example of the drug in emulsified state is shown in FIG. 1. The drug is present in W phase while being dissolved in ethanol (E), the oil forms O phase (O), followed by emulsification through homogenization as shown in the right side of FIG. 1.

Next, a step of extracting under supercritical or subcritical conditions is performed (S2).

At this time, the supercritical conditions mean that a fluid state rather than a liquid or gas under conditions of critical temperature and critical pressure or more, while the subcritical conditions mean that it exceeds saturated vapor pressure under a condition of less than the critical temperature.

The above step S2 may be performed by extracting the solvent using a supercritical or subcritical extraction apparatus.

When the step S2 is performed under the supercritical condition, after adding the product in the previous step S1 and an additive to the supercritical fluid and dissolving the same in the supercritical fluid, the mixture may be vented thus to extract the solvent. At this time, the supercritical fluid used herein may be a carbon dioxide fluid at room temperature. Preferably, a temperature of the carbon dioxide ranges from 30 to 32° C., and more preferably, 31° C. When the step S2 is implemented under the supercritical condition, a range of the supercritical pressure may be 1000 to 6000 psi (68 to 400 bar). The additive may include excipients. The excipients may include pharmaceutically acceptable disintegrants, binders, fillers and lubricants. The disintegrant may include, for example, agar-agar, algin, calcium carbonate, carboxymethyl cellulose, cellulose, clay, colloidal silicon dioxide, croscarmellose sodium, crospovidone, rubber, magnesium aluminum silicate, methyl cellulose, polacrylin potassium, sodium alginate, low-substituted hydroxypropyl cellulose, and cross-linked polyvinyl pyrrolidone hydroxypropyl cellulose, sodium starch glycolate and starch. The binder may include, for example, microcrystalline cellulose, hydroxylmethyl cellulose, hydroxypropyl cellulose and polyvinyl pyrrolidone. The filler may include, for example, calcium carbonate, calcium phosphate, 2-basic calcium phosphate, 3-basic calcium sulfate, calcium carboxymethyl cellulose, cellulose, dextrin derivatives, dextrin, dextrose, fructose, lactitol, lactose, magnesium carbonate, magnesium oxide, maltitol, maltodextrin, maltose, sorbitol, starch, sucrose, sugar and xylitol. The lubricant may include, for example, agar, calcium stearate, ethyl oleate, ethyl laurate, glycerin, glyceryl palmitostearate, hydrogenated vegetable oil, magnesium oxide, magnesium stearate, mannitol, poloxamer, glycol, sodium benzoate, sodium lauryl sulfate, sodium stearyl, sorbitol, stearic acid, talc and zinc stearate.

According to an embodiment of the present invention, the nanoemulsion may be prepared through emulsification in the previous step S1, and the present step S2 may be performed by appropriately adjusting each content of constitutional ingredients and then inputting the same into a supercritical carbon dioxide chamber, wherein the inputting process may be selected from an injecting manner through nozzles or a method of inputting from a lower part of the chamber. After inputting the product of the previous step S1 into supercritical carbon dioxide, nanosization may be performed through reduced pressure filtration. Further, in consideration of major roles of the supercritical fluid in a particle formation process, that is, a role of solvent, a role of anti-solvent or reverse-solvent, and a role of additive-solute, any of appropriate reaction methods for particle formation may be selected.

The method of producing nanoparticles according to an aspect of the present invention may use the compound represented by Formula 1 as a drug to be nanosized, wherein this compound does not have a high solubility to a supercritical fluid, and therefore, a supercritical fluid method through rapid expansion is hardly applicable. Instead, specific and applicable methods may include, for example, an aerosol solvent extraction system (ASES) process or a solution enhanced dispersion by supercritical fluids process (SEDS) among supercritical anti-solvent (SAS) processes which are methods of acquiring re-crystals using a supercritical fluid as a reverse-solvent.

When performing the present step S2 under the subcritical conditions, venting and extraction may be executed in a subcritical fluid after dissolution, which are similar to the above-described supercritical conditions. At this time, considering that the drug for treating hepatitis has a melting point of 102° C., high pressure subcritical water at a temperature of 110 to 140° C., and preferably, 130° C., which is higher than the melting point, may be used as the subcritical fluid. In this case, the pressure of the subcritical water may range from 4 to 400 bar at a temperature of 100 to 374° C. After dissolving the subcritical fluid, the solution may be subjected to high speed agitation and vacuum extraction to perform nanorization. Further, a size of the prepared particles may be regulated by adjusting conditions for vacuum extraction.

When nanoparticles are synthesized at room temperature or under conditions for general hydrothermal synthesis, a reaction time is delayed which in turn causes problems in that it is difficult to control a particle size and it is difficult to homogenously proceed a surface modification reaction. The production method according to an aspect of the present invention may synthesize nanoparticles under supercritical or subcritical conditions to reduce surface energy of a particle precursor while increasing a diffusion rate of a solvent so that the reaction is performed in a short time, whereby problems of the conventional art can be overcome and the prepared nanoparticles may be enhanced in terms of a size and surface modification homogeneity.

Next, a step of dispersing the product obtained in the previous step S2 may be implemented (S3).

The present step S3 may be performed by water dispersion using a high pressure homogenizer. Through this, the drug for treating hepatitis and excipients may be dispersed.

For the dispersed nanoparticles, purification may be performed by applying at least one process selected from the group consisting of dialysis and free-drying, whereby final nanoparticles according to an aspect of the present invention can be produced.

The nanoparticles produced by the method according to an aspect of the present invention may be formed to have a uniform size along with a valid average particle size of 500 to 800 nm and a deviation in particle sizes in a range of 0 to 50 nm. Further, the produced nanoparticles are not easily coagulated due to excellent surface stability, and are removed without residues of the organic solvent during processing, thereby exhibiting excellent features for application to a pharmaceutical composition.

Further, the surfactant is adsorbed in an amount enough to maintain the valid average particle size of less than 1000 nm on the surface of nanosized compound and formed as the surface modifier. The adsorbed surfactant does not chemically react between the surfactant-surfactant and between the surfactant-medical compound, thereby forming stabilized particles. Furthermore, the adsorbed surfactant molecules have very little cross-linkage formed between molecules.

The method of producing nanoparticles according to an aspect of the present invention may also be applicable even when using different poorly water-soluble drugs as a subject for nanorization. Examples of the poorly water-soluble drugs to which the production method of the present invention is applicable may include at least one selected from the group consisting of analgesic agents, anti-inflammatory agents, anti-depressants, antibiotics, anti-hypertensive agents, back pain analgesic agents, antiphlogistics, anthelmintics, antiarrhythmics, antibiotics (including penicillin), anti-coagulants, anti-anxiety agents, anti-diabetics, anti-epileptics, antihistaminic agents, anti-hypertensive agents, anti-muscarinic agents, anti-mycobacterial agents, antitumor agents, immuno-suppressants, antithyroidal agents, antiviral agents, sedatives (somnifacients and neuroleptics), astringents, beta-adrenergic receptor blockers, blood products and substitutes, cardiopulmonary agents, contrast agents, corticosteroids, cough suppressants (expectorants and mucolytics), diagnostic agents, diagnostic imaging agents, diuretics, dopamine agonists (therapeutic agents for Parkinson's disease), hemostatic agents, immunological agents, lipid modulating agents, muscle relaxants, parasympathetic stimulants, parathyroid calcitonin and biphosphonate, prostaglandins, radiopharmaceuticals, sex hormones (including steroids), antiallergics, anti-stimulants and antianoretics, sympathetic stimulants, thyroidal agents and vasodilators.

Hereinafter, in order to facilitate understanding of the present invention, the present invention will be described in more detail with reference to the following examples. However, these examples are only provided for illustrating the present invention and are not intended to limit the appended claims. Therefore, it is obviously understood by those skilled in the art that various alternations and modifications of the examples are possible within the scope of the technical spirit of the present invention. Further, such modifications and alterations should be construed as being included in the appended claims.

### Example

<Preparation of Precursor Mixture Solution for Nanoparticles Dissolved in Ethanol>

A mixture solution was prepared by mixing a poorly water-soluble drug, a surface modifier and a stabilizer. The stabilizer used herein is polyvinyl pyrrolidone (PVP; Kollidon® 17PF), and 1-hexadecanol was also used as a lipid. A mixing ratio of the poorly water-soluble drug:surface modifier:stabilizer was A:B:C, and the mixture was heated while stirring until the above ingredients were completely dissolved, followed by cooling the same at room temperature for 24 hours so as to solidify the same. As the poorly water-soluble drug, each one selected from the group consisting of Daclatasvir, Grazoprevir, Asunaprevir and Velpatasvir was selectively used. Therefore, for the selected drugs, a total 4 types of precursor mixture solutions were prepared.

<Preparation of Precursor Mixture Solution for Synthesis of Nanoparticles in Emulsion State>

According to an emulsification method so that a poorly water-soluble drug, oil for formation of an oil phase, ethanol for formation of a water phase, and a derivative including a surfactant are efficiently oriented on an interface to reduce a particle size, a nanoemulsion was prepared.

The preparation of emulsion was performed by heating the oil phase and the water phase to 80° C. to completely dissolve the same, conducting pre-emulsification at H/M 2,000 rpm while slowly introducing ethanol as the water phase to the oil phase, followed by secondary emulsification at H/M 5,000 rpm for 10 minutes and cooling the product. A content of the oil used for experiment was 10 to 30%, and the next day after preparation, stability was measured. Then, the product was stored at 25° C. and 45° C., respectively, followed by determining the stability of emulsion. It was observed that the stability of emulsion between the drug/surfactant is sufficient during extraction, and therefore, it was confirmed that the product has high reliability and is appropriately applicable to an extendable process.

<Extraction Using Supercritical Fluid>

Critical temperatures and critical pressures of the supercritical fluids are shown in Table 1 below.

TABLE 1

| | Critical temperature | Critical pressure |
|---|---|---|
| | (° C.) | (MPa) |
| H₂O | 374.0 | 22.0 |
| Xe | 16.6 | 5.9 |
| SF₆ | 45.5 | 3.8 |
| N₂O | 36.5 | 4.1 |
| C₂H₄ | 9.1 | 5.1 |
| CHF₃ | 25.9 | 4.7 |
| CO₂ | 31.3 | 7.4 |

The supercritical carbon dioxide has a critical temperature at room temperature which is a low temperature, as shown in Table 1. The supercritical water has a high temperature of 300° C. or more, and is hardly used since it may thermally decompose the drug. Further, since Xe, SF₆, N₂O, C₂H₄ and CH₃ are hardly applicable due to economical disadvantages or harmfulness, the supercritical carbon dioxide was selected as a solvent for extraction according to a supercritical method.

From each of precursors prepared in the previous steps, the poorly water-soluble drug prepared by dissolving it in ethanol, or the poorly water-soluble drug prepared by nano-emulsification using ethanol as a water phase, lipid was removed using supercritical carbon dioxide by means of a supercritical extraction apparatus, followed by forming particles.

Specifically, carbon dioxide was introduced in a flow rate of 12 g/min at 31° C. and 100 atm into a sedimentator having a volume of 34 cm³ and then 0.8 wt. % of the precursor solution was injected at a flow rate of 0.78 ml/min into the sedimentator through nozzles. After completion of the injection, the solution was washed using pure carbon dioxide, followed by reducing the pressure in order to recover microparticles. The particles were prepared using ethanol as a solvent.

The particles remaining after removal of the lipid was dispersed in an additive solution by a high pressure homogenizer process and dried through dialysis and freeze-drying, thereby acquiring final nanoparticles for each of the compounds.

<Extraction Using Subcritical Fluid>

15 ml of the precursor solution was introduced into 50 ml high temperature and pressure reactor made of stainless steel (SUS 316). The reactor was placed in a salt bath at 130° C., and was subjected to a reaction under a pressure of 300 bar. After the reaction, the reactor was chilled with cold water while recovering nanoparticles from the completely reacted solution using a filter. Unreacted lipid was removed by a process of dissolving it in ethanol and then washing out the same, which was repeated three times. The washed nanoparticles entered a vacuum oven at 80° C. and were dried for 24 hours to remove the ethanol, thereby remaining the particles. The remaining particles were dispersed in an additive solution by a high pressure homogenizer process and dried through dialysis and freeze-drying, thereby acquiring final nanoparticles for each of the compounds.

<Confirmation of Physical Properties of the Produced Nanoparticles>

With regard to the produced nanoparticles, morphology analysis through a scanning electronic microscope, investigation of crystallinity through powdery X-ray diffraction, and confirmation of residual solvent through gas chromatography according to the general test methods of the Korean Pharmacopeia were performed to determine features of the nanoparticles.

For morphology analysis, each solid was fixed on a sample stand using a carbon tape, and coated with platinum (Pt) using an ion coater. As a result of photographing the sample by the scanning electron microscope (SEM), it was confirmed that particles having an average volume diameter in a range of 100 to 1000 nm were consistently produced. The produced nanoparticles exhibited excellent consistency in terms of the particle size.

In order to confirm a crystalline structure of the particles, an experiment was conducted using an X-ray diffractometer. At this time, each analysis result was obtained from measurement in a region of 3 to 50° (2θ) at a rate of 3° per minute.

Further, according to the elution test method No. 2 (paddle method) among the general test methods of the Korean Pharmacopoeia, an experiment for elution rate was performed. Specifically, after dissolving the nanoparticles in 900 ml of phosphate buffer with pH 5.8 at 37° C., and then agitated at a rate of 50 rpm. On every 1, 2, 5, 10, 20, 30, 60, 120 minutes, 10 mL of solution was taken and filtered using a membrane filter (pore size: 0.5 μm), followed by measuring absorbance at 243 nm using a UV-VIS spectrometer. As a result of the measurement, it was confirmed that the elution rate was improved thus to exhibit excellent solubility.

<Confirmation of Therapeutic Effects of Nanosized Poorly Water-Soluble Drug>

Effects of hepatitis treatment by the produced nanoparticles were confirmed through animal experiments. For effective treatment, with regard to the drugs known to be useful for combined administration, a content of other drugs administered in combination was secured while adjusting a content of the nanosized poorly water-soluble drug in order to investigate therapeutic effects thereof. As a result of the investigation, it was confirmed that the nanosized poorly water-soluble drug exhibits excellent effects with a lower dose reduced compared to a content of the conventional non-nanosized drug. The reduced dose is different depending on medical compounds and it was confirmed that, even when administered in the dose decreased by 15 to 40% in weight compared to the conventional non-nanosized drug, the above nanosized poorly water-soluble drug exhibits therapeutic effects.

Major parameters in relation to adjustment of the particle sizes include a size of emulsion droplets, a concentration of drug solution and a content of organic solvent. Extraction was effectively and rapidly performed to a level of microgram (μg), and the nanoparticles produced thereby were essentially crystalline and showed an increase of 5 to 10 times in terms of a dissolution rate of an effective drug as compared to simple microfine powder. According to theoretical calculation, the dissolution depends on a surface motion coefficient, and a specific surface area of the produced particles.

With regard to the nanoparticles produced according to the examples of the present invention, all of four (4) compounds have a water solubility of 2 mg/mL or more, thereby demonstrating excellent results. Accordingly, the nanoparticles of the present invention may be applicable as a pharmaceutical composition for treating hepatitis with excellent bioavailability.

What is claimed is:

1. A method of producing nanoparticles for treating hepatitis, the method comprising:
a first step of preparing a precursor mixture solution for synthesis of nanoparticles which comprise any one of compounds represented by Formulae 1 to 4 below: 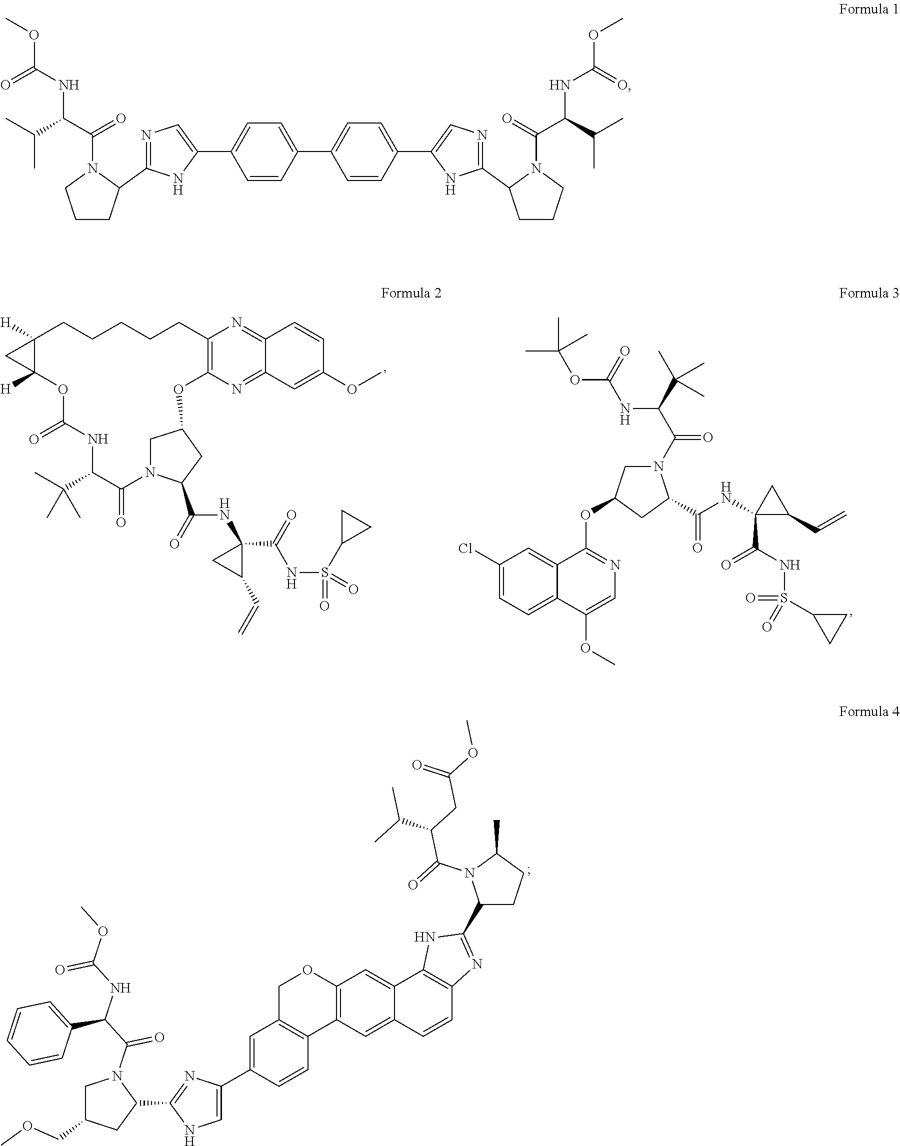
a second step of extracting the compound under supercritical or subcritical conditions; and
a third step of dispersing the same.

2. The method according to claim 1, wherein the precursor mixture solution is a nanoemulsion or microemulsion comprising any one of the compounds represented by Formulae 1 to 4.

3. The method according to claim 2, wherein the nanoemulsion or microemulsion is prepared by mixing any one of the compounds represented by Formulae 1 to 4, oil, a surfactant and alcohol,
wherein the oil, the surfactant and the alcohol are included in a weight ratio value of 1 to 10 according to Equation 1 below, respectively:
(Weight ratio)=(A)/[(B)+(C)] (Equation 1)
wherein in Equation 1 above, A represents a weight of the oil, B represents a weight of the surfactant, and C represents a weight of the alcohol.

4. The method according claim 1, wherein the precursor mixture solution is prepared by mixing any one of the compounds represented by Formulae 1 to 4, a surface modifier and a stabilizer in a solvent.

5. The method according to claim 4, wherein the precursor mixture solution is sequentially heated and cooled to produce a solid lipid matrix.

6. The method according to claim 1, wherein the second step is performed by adding the product prepared in the first step and an additive to a supercritical carbon dioxide fluid at 30 to 32° C., dissolving the same in the supercritical carbon dioxide fluid, and then extracting the same.

7. The method according to claim 1, wherein the second step is performed by adding the product prepared in the first step and an additive to a subcritical water fluid at 110 to 140° C., dissolving the same in the subcritical water, and then extracting the same.

8. Nanoparticles for treating hepatitis, which are produced by the production method according to claim 1.

9. Nanoparticles for treating hepatitis, which are produced by the production method according to claim 2.

10. Nanoparticles for treating hepatitis, which are produced by the production method according to claim 3.

11. Nanoparticles for treating hepatitis, which are produced by the production method according to claim 4.

12. Nanoparticles for treating hepatitis, which are produced by the production method according to claim 5.

13. Nanoparticles for treating hepatitis, which are produced by the production method according to claim 6.

14. Nanoparticles for treating hepatitis, which are produced by the production method according to claim 7.
